(19) United States
(12) Patent Application Publication
(10) Pub. No.: US 2007/0122350 A1
(43) Pub. Date: May 31, 2007

(54) Safe and effective methods of administering therapeutic agents

(75) Inventors: Frank G. Pilkiewicz, Princeton, NJ (US); Walter Perkins, Pennington, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: Transave, Inc., Monmouth Junction, NJ (US)

(21) Appl. No.: 11/606,392

(22) Filed: Nov. 29, 2006

Related U.S. Application Data
(60) Provisional application No. 60/740,696, filed on Nov. 30, 2005.

(51) Int. Cl. A61K 9/12; A61K 9/127; A61K 33/24; A61K 31/282

(52) U.S. Cl. 424/45; 424/450; 424/649; 514/492

(57) ABSTRACT

In one embodiment, the invention relates to a kit for administering a lipid-based platinum compound formulation via inhalation comprising: an inhalation device comprising a lipid-based platinum compound formulation, a demistifier tent of sufficient size to at least cover a patient's head and the inhalation device, and instructions for use thereof. In another embodiment, the invention relates to a methods of administering therapeutics and increasing safety of administering therapeutics thereof. In another embodiment, the invention relates to an inhalation device for administering a therapeutic agent to a patient comprising a mouth piece, a nebulizer, and a holder for preventing the patient's saliva from entering the nebulizer or returning to the patient during administration.

### RELATED APPLICATIONS

This application claims the benefit of priority to U.S. Provisional Patent Application Ser. No. 60/740,696, filed Nov. 30, 2005, which is hereby incorporated by reference in its entirety.

### FIELD OF INVENTION

The present invention relates to the administration of therapeutic agents and kits and methods thereof.

### BACKGROUND OF THE INVENTION

Cis-diamminedichloroplatinum(II) (Pt(NH₃)₂Cl₂), better known as cisplatin, is a divalent inorganic water-soluble, platinum-containing complex with antineoplastic activity. A major cytotoxic mode of action is binding to DNA and interference with the cell's repair mechanism. The drug is used in therapy against a variety of neoplasms including lung cancer. One of the most important disadvantages of cisplatin is its toxicity profile, e.g. without precautions like forced hydration, severe renal toxicity is seen. Safirstein, R. L. Renal diseases induced byanti-neoplastic agents. In R. W. Schrier (ed.), Diseases of the kidney and urinary tract., 7 ed, pp. 1175-1188. Philadelphia: Lippincott Williams & Wilkins, 2001. Furthermore, cisplatin is one of the most highly ematogenic agents in cancer chemotherapy. Hesketh, P. J. et al., J. Clin. Oncol., 15: 103-109, 1997.

Recently, a sustained release, reduced toxicity formulation of cisplatin has been developed. See U.S. Pat. No. 6,793,912; U.S. Published Patent Application Nos. 2005/0107287 A1; 2004/0101553 A1; 2003/0059375 A1; and U.S. patent application Ser. Nos. 11/084,070; 11/135,625; the entirety of which are incorporated herein. The cisplatin is encapsulated inside nano-scale liposomal carriers and administered to the patient via nebulization. This new way of administration of cisplatin is currently under investigation in a phase I trial. The idea is to improve the therapeutic index of the antineoplastic agent by delivering higher concentrations at the tumor site, i.e., directly to the lung by inhalation, thus improving efficacy while avoiding systemic toxicities. Because inhaled cytotoxic agents without a specific carrier such as cisplatin are rapidly cleared from the lung, a liposomal encapsulated formulation of cisplatin, can be used to maintain drug in the lung and provide sustained release. Additionally, due to the particulate nature of liposomes, lymphatic absorption will be increased which may be a benefit for lymphangenic spread of metastatic disease.

Due to the cytotoxic properties of cisplatin, it is necessary to prevent drug exposure to the health care workers (HCW). For this reason, during administration of lipid-based platinum compound formulations, HCW are dressed in full barrier protection clothing (safety glasses, breathing mask, gown, gloves, cap, and sleeves), treatment is performed in a negative pressure room, and the drug is administered to the patients inside a demistifier tent (for example, the Peace Medical Demistifier, model 2000). The demistifier tent isolates the patient in a vinyl enclosure similar to an oxygen tent. Outside air is drawn upward into the tent and then from the area inside the canopy it flows through a HEPA system at a rate of 240 to 360 air changes per hour. Previous studies with aerosolized toxic agents like ribavirin (used to treat severe respiratory syncytial virus infections in infants and young children), and pentamidine (treatment of or prophylaxis against Pneumocystis carinii in immune compromised patients/persons) demonstrated the effectiveness of the demistifier tent in limiting occupational exposure. Wahlin, B. et al., Acta Anaesthesiol. Scand., 40: 932-939, 1996; Krilov, L. R. Pediatr. Infect. Dis. J, 21: 479-481, 2002; Adams, D. A. Environmental Exposures of Health Care Personnel to Aerosolized Ribavirin: Nursing Implications. Worldviews on Evidence-Based Nursing, el: 79-87, 1994; Weinthal, J. et al. J. Clin. Oncol., 12: 136-140, 1994. The importance of using a demistifier was also stated in an aerosol consensus report regarding special considerations of caregiver protection during and directly after aerosol treatment with pentamidine or ribavirin. Aerosol consensus statement. Consensus Conference on Aerosol Delivery. Chest, 100: 1106-1109, 1991; Aerosol consensus statement-1991. American Association for Respiratory Care. Respir. Care, 36: 916-921, 1991.

In addition to HCW safety, there is also a problem for the patient undergoing treatment. Inhalation treatment is done over a period time wherein the patient is continuously breathing from an inhalation device. During this time, some of the patient's saliva may collect in the device near the mouth piece. The saliva will then flow into the nebulizer and mix with drug yet to be administered. In this situation, the saliva will adversely impact the proper nebulization and delivery of the remaining drug. The time to nebulize the remaining drug mixture will be prolonged, thus adversely impacting patient comfort and possibly altering the nebulized characteristics of the drug.

Although administration by inhalation has many advantages, there still exists a need for improved safety and comfort for all involved, especially when platinum compound formulations are involved.

### SUMMARY OF THE INVENTION

It is an object of the present invention to improve the safety and comfort of those who are involved with the administration of therapeutic agents by inhalation techniques. The safety of HCW is especially important when the therapeutic agent is a cytotoxic platinum compound formulation. Dosing of drug is important and must be done properly without compromise to the drug or increase in nebulization time.

The subject invention results from the realization that kits comprising a demistifier tent and an inhalation device containing a lipid-based platinum compound formulation offer a convenient way for hospital workers and other HCW to safely administer lipid-based platinum compound formulations to a patient in need thereof. The subject invention also results from the realization that a holder placed in close proximity to the mouth piece of an inhalation device prevent's a patient's saliva from entering the nebulizer, preventing contamination and prolongation of treatment. The holder may be in the form of a section of tubing placed between the mouth piece and exhalation filter of most existing inhalation devices, or it may be built into the inhalation device as a cup in close proximity to the mouth piece.

In one embodiment, the present invention relates to a kit for administering a lipid-based platinum compound formulation via inhalation comprising: an inhalation device comprising a lipid-based platinum compound formulation, a demistifier tent of sufficient size to at least cover a patient's head and the inhalation device, and instructions for use thereof. In a further embodiment the kit also comprises a stand to hold the inhalation device.

In a further embodiment, the invention relates to the aforementioned kit wherein the platinum compound is selected from the group consisting of: cisplatin, carboplatin (diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof. In a further embodiment, the platinum compound is cisplatin.

In a further embodiment, the present invention relates to the aforementioned kit wherein the lipid-based platinum compound formulation comprises a liposome. In a further embodiment, the liposome has a mean diameter of 0.01 microns to 3.0 microns.

In a further embodiment, the present invention relates to the aforementioned kit wherein the lipid is a mixture of a phospholipid and sterol. In a further embodiment, the lipid is selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In a further embodiment, the lipid is a mixture of DPPC and cholesterol. In a further embodiment, the lipid is a mixture of DPPC from 50 to 65 mol % and cholesterol from 35 to 50 mol %.

In a further embodiment, the present invention relates to the aforementioned kit wherein the demistifier tent is large enough to cover the patient's head and inhalation device. In a further embodiment, the demistifier is large enough to contain the patient.

In a further embodiment, the present invention relates to the aforementioned kit wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

In a further embodiment, the present invention relates to the aforementioned kit wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

In another embodiment, the present invention relates to a method of administering a lipid-based platinum compound formulation via inhalation to a patient in need thereof comprising administering the formulation to the patient while the patient is partially or wholly within a demistifier tent.

In a further embodiment, the present invention relates to the aforementioned method wherein the platinum compound is selected from the group consisting of: cisplatin, carboplatin (diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof. In a further embodiment, the platinum compound is cisplatin.

In a further embodiment, the present invention relates to the aforementioned method wherein the lipid-based platinum compound formulation comprises a liposome. In a further embodiment, the liposome has a mean diameter of 0.01 microns to 3.0 microns.

In a further embodiment, the present invention relates to the aforementioned method wherein the lipid is a mixture of a phospholipid and sterol. In a further embodiment, the lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1, 2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In a further embodiment, the lipid is a mixture of DPPC and cholesterol. In a further embodiment, the lipid is a mixture of DPPC from 50 to 65 mol % and cholesterol from 35 to 50 mol %.

In a further embodiment, the present invention relates to the aforementioned method wherein the demistifier tent is large enough to cover the patient's head and inhalation device. In a further embodiment, the demistifier is large enough to contain the patient.

In a further embodiment, the present invention relates to the aforementioned method wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

In a further embodiment, the present invention relates to the aforementioned method wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

In another embodiment, the present invention relates to a method of increasing the safety of administering a lipid-based platinum compound formulation via inhalation for a person comprising administering the formulation to a patient while the patient is partially or wholly within a demistifier tent.

In a further embodiment, the present invention relates to the aforementioned method wherein the platinum compound is selected from the group consisting of: cisplatin, carboplatin (diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof. In a further embodiment, the platinum compound is cisplatin.

In a further embodiment, the present invention relates to the aforementioned method wherein the lipid-based platinum compound formulation comprises a liposome. In a further embodiment, the liposome has a mean diameter of 0.01 microns to 3.0 microns.

In a further embodiment, the present invention relates to the aforementioned method wherein the lipid is a mixture of a phospholipid and sterol. In a further embodiment, the lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1, 2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In a further embodiment, the lipid is a mixture of DPPC and cholesterol. In a further embodiment, the lipid is a mixture of DPPC from 50 to 65 mol % and cholesterol from 35 to 50 mol %.

In a further embodiment, the present invention relates to the aforementioned method wherein the demistifier tent is large enough to cover the patient's head and inhalation device. In a further embodiment, the demistifier is large enough to contain the patient.

In a further embodiment, the present invention relates to the aforementioned method wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

In a further embodiment, the present invention relates to the aforementioned method wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

In another embodiment, the present invention relates to an inhalation device for administering a therapeutic agent to a patient comprising a mouth piece, a nebulizer, and a holder for preventing the patient's saliva from entering the nebulizer or returning to the patient during administration. In a further embodiment, the inhalation device further comprises an exhalation filter. In a further embodiment, the holder is a section of tubing placed between the mouth piece and the exhalation filter such that the section of tubing slopes downwards and then upwards, or the section of tubing slopes downwards and then levels out to be substantially parallel to the ground. In a further embodiment, the holder is a cup placed between the mouth piece and the nebulizer. In a further embodiment, the cup has a removable bottom for emptying the saliva.

In a further embodiment, the present invention relates to the aforementioned device wherein the therapeutic agent comprises a chemotherapeutic agent. In a further embodiment, the therapeutic agent comprises a platinum compound, antiviral compound, antibacterial compound, or antifungal compound. In a further embodiment, the platinum compound is selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof. In a further embodiment, the therapeutic agent comprises cisplatin.

In a further embodiment, the present invention relates to the aforementioned device wherein the therapeutic agent comprises a lipid-based platinum compound formulation. In a further embodiment, the platinum compound is cisplatin.

In a further embodiment, the present invention relates to the aforementioned device wherein the lipid-based platinum compound formulation comprises a liposome. In a further embodiment, liposome has a mean diameter of 0.01 microns to 3.0 microns.

In a further embodiment, the present invention relates to the aforementioned device wherein the lipid is a mixture of a phospholipid and sterol. In a further embodiment, the lipid is selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1, 2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof. In a further embodiment, the lipid is a mixture of DPPC and cholesterol. In a further embodiment, the lipid is a mixture of DPPC from 50 to 65 mol % and cholesterol from 35 to 50 mol %.

In a further embodiment, the present invention relates to the aforementioned device wherein the lipid-based platinum compound formulation comprises a liposome having a mean diameter of 0.01 microns to 3.0 microns, and cisplatin, and wherein the lipid is a mixture of DPPC from 50 to 65 mol % and cholesterol from 35 to 50 mol %.

These embodiments of the present invention, other embodiments, and their features and characteristics, will be apparent from the description, drawings and claims that follow.

### BRIEF DESCRIPTION OF THE DRAWINGS

FIG. 1 depicts the experimental set up used in the Exemplification section to administer lipid-based cisplatin formulations within a demistifier tent.

FIG. 2 depicts a standard curve of the measured platinum concentration by ICP-MS. The slope of the standard curve, (S)=1.038, as determined by linear regression.

FIG. 3 depicts a patient within a demistifier tent large enough to contain the patient showing the flow of air from entrance to exit through a HEPA filter.

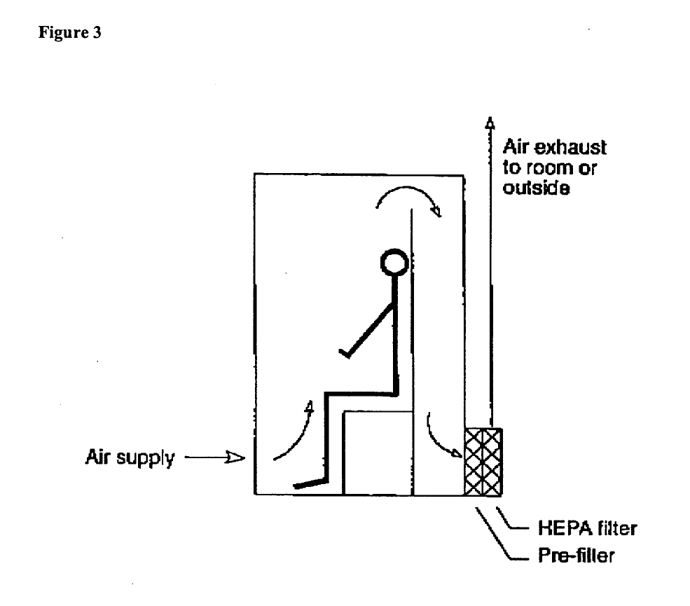

FIG. 4 depicts a blown up schematic view of a common type of inhalation device comprising a mouth piece, exhalation filter, and nebulizer.

FIG. 5 depicts a blown up schematic view of the same inhalation device from FIG. 4 except a section of flexible tubing is situated between the mouth piece and exhalation filter. The flexible tubing is first positioned downward as it leaves the y-connector and then curves to the side and upwards such that the exhalation filter is disposed sideways relative to the mouth piece and nebulizer such that saliva running into the tubing will not collect in the exhalation filter or enter the nebulizer.

FIG. 6 depicts a photograph of the inhalation device from FIG. 4 showing the position of the flexible tubing and exhalation filter relative to the mouth piece and nebulizer. In this embodiment the flexible tubing first points downward and then curves upward.

FIG. 7 depicts a photograph of the inhalation device from FIG. 4 showing the position of the flexible tubing and exhalation filter relative to the mouth piece and nebulizer. In this embodiment the flexible tubing first points downwards and then levels out to be substantially parallel to the ground.

FIG. 8 depicts one embodiment of the inhalation device where the holder for collecting a patient's saliva is in the form of a cup between the mouth piece and exhalation filter.

FIG. 9 depicts a stand as a part of one embodiment of the kits of the present invention.

### DETAILED DESCRIPTION OF THE INVENTION

I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “bioavailable” is art-recognized and refers to a form of the subject invention that allows for it, or a portion of the amount administered, to be absorbed by, incorporated to, or otherwise physiologically available to a subject or patient to whom it is administered.

The term “cancer treating effective amount” as used herein refers to the amount of lipid-based platinum compound formulation effective for the treatment of cancer. In one embodiment the cancer treating effective amount of lipid-based platinum compound formulation is typically about 100 mg/m² for a human.

The terms “comprise” and “comprising” are used in the inclusive, open sense, meaning that additional elements may be included.

The term “HEPA” stands for High Efficiency Particulate Air is used in conjunction with a filter air cleaning system. A “HEPA-filter air cleaning system” is used interchangeably with the term “demistifier.”

The term “hydrophobic matrix carrying system” is a lipid/solvent mixture prepared during the solvent infusion process described below.

The term “including” is used herein to mean “including but not limited to”. “Including” and “including but not limited to” are used interchangeably.

The terms “lipid-based platinum compound” as used herein refers to a composition comprising a lipid and a platinum compound. In some embodiments the lipid-based platinum compound can be in the form of a liposome. In some embodiments the ratio of platinum compound to lipid in the lipid-based platinum compound can be between about 1:5 by weight and 1:50 by weight. In a further embodiment, the ratio of platinum compound to lipid in the lipid-based platinum compound can be between about 1:5 and about 1:30. In a further embodiment, the ratio of platinum compound to lipid in the lipid-based platinum compound can be between about 1:5 by weight and 1:25 by weight. In still other embodiments, the platinum compound can be cisplatin.

The term “mammal” is known in the art, and exemplary mammals include humans, primates, bovines, porcines, canines, felines, and rodents (e.g., mice and rats).

A “patient,” “subject” or “host” to be treated by the subject method may mean either a human or non-human animal.

The term “pharmaceutically-acceptable salts” is art-recognized and refers to the relatively non-toxic, inorganic and organic acid addition salts of compounds, including, for example, those contained in compositions of the present invention.

The term “slopes downwards” as used herein refers to the disposition of the section of tubing located between a mouth piece and an exhalation filter of the inhalation device of the present invention. The section of tubing points downward but then curves upward towards the exhalation filter. This position allows the patient's saliva to collect in the tubing without contaminating the exhalation filter or returning to the patient during administration.

The term “solvent infusion” is a process that includes dissolving one or more lipids in a small, preferably minimal, amount of a process compatible solvent to form a lipid suspension or solution (preferably a solution) and then adding the solution to an aqueous medium containing bioactive agents. Typically a process compatible solvent is one that can be washed away in a aqueous process such as dialysis. The composition that is cool/warm cycled is preferably formed by solvent infusion. Alcohols are preferred as solvents, with ethanol being a preferred alcohol.

“Ethanol infusion,” is a type of solvent infusion that includes dissolving one or more lipids in a small, preferably minimal, amount of ethanol to form a lipid solution and then adding the solution to an aqueous medium containing bioactive agents. A “small” amount of solvent is an amount compatible with forming liposomes or lipid complexes in the infusion process.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic index” is an art-recognized term which refers to the ratio of a quantitative assessment of toxicity to a quantitative assessment of efficacy of a drug, e.g. LD₅₀/ED₅₀ in the case of animals. The term “LD₅₀” is art recognized and refers to the amount of a given toxic substance that will elicit a lethal response in 50% of the test organisms. This is sometimes also referred to as the median lethal dose. The term “ED₅₀” is art recognized and refers to the median effective dose.

The term “treating” is art-recognized and refers to curing as well as ameliorating at least one symptom of any condition or disease.

II. Safe Methods and Kits for Administering Lipid-Based Platinum Compound Formulations

Methods and kits for safely administering lipid-based platinum compounds to a patient in need thereof resulted from ongoing treatments of patients and experiments to determine platinum levels within the demistifier tent environment. The following describes the experiments conducted and discusses the results obtained.

II.A. Experimental Setup

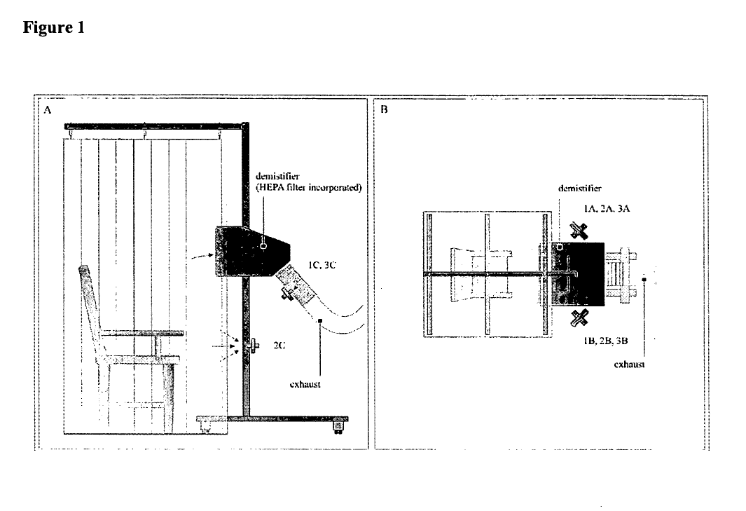

Air sampling was conducted under three different sets of conditions. The first condition was performed during patient dosing, i.e. while patients were in the tent during treatment. Air was sampled cumulatively over numerous 1-hour sessions of patient treatment. Two air-sampling cartridges were placed around the outside of the tent (see 1A and 1B in FIG. 1) and one was placed in the exhaust stream of air from the tent (see 1C in FIG. 1). FIG. 1 shows a side and top view of the experimental set up with the locations of the filters during the investigation. The second condition was performed where another set of three cartridges (labeled 2A, 2B, and 2C in FIG. 1) sampled the air outside the tent for a period of time directly following completion of patient dosing, after the patient had left the tent (a portion of the tent's plastic canopy must be raised in order for the patient to leave the tent). Patients remain seated in the tent the first five minutes after the inhalation has been completed to allow for the clearance of aerosol. Sampling was cumulative as well, and the three cartridges were placed similar to that used in condition one, except the third cartridge was not used to sample the exhausted air but rather was placed outside the tent near the other two cartridges. The exhaust air was of little concern at this point since no more aerosol was being generated inside the tent. The third condition was performed without a patient present in the tent. A nebulizer with 7 mL of drug product was run in the tent for over 20 minutes, to dryness. The three cartridges (3A, 3B, and 3C in FIG. 1) were located in the same positions as those for condition one. Prior to initiation of the study, the airflow of each filter cartridge was sampled. The airflow values of 3A, 3B, and 3C were assumed to be the same values as those measured for cartridges 1A, 1B, and 1C, respectively. To record data from the sampling during patient treatment, a log sheet was used to track air sampling times and conditions. Data from this log contained information about the patient present during measurements, the dates of the measurements, and the sampling time during and after the treatment (stages 1 and 2, consecutively).

II.B. Assay of Filters for Platinum

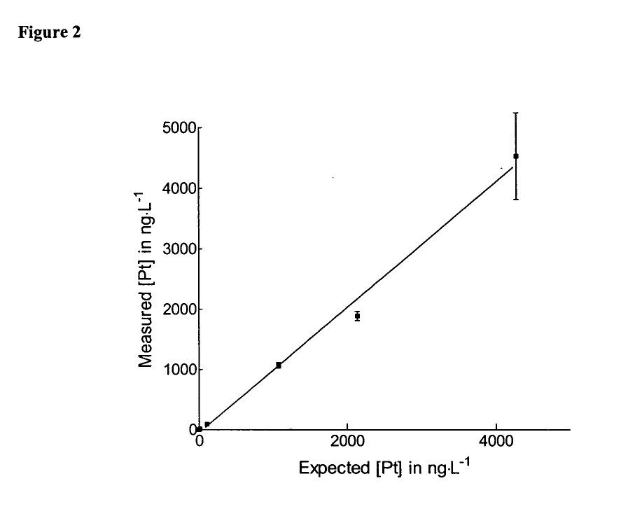

To evaluate the analytical procedure for extraction and quantitation of platinum by Inductively Coupled Plasma-Mass Spectrometry (ICP-MS), standard concentrations were prepared for lipid-based cisplatin formulations that had been deposited onto the same filters as those used for the air sampling, and the solutions of free cisplatin or lipid-based cisplatin. For preparation of standards with lipid-based cisplatin, a concentration of 9.99×10⁻¹ mg/ml was utilized and was further diluted to 9.99×10⁻² and 9.99×10⁻³ mg/ml in the preparation of a standard curve (Table 1 and FIG. 2).

TABLE 1
Evaluation of ICP-MS in measuring platinum content
in solution extracts of 18 control filter samples
of lipid-based cisplatin formulations.

Pt levels (ng/liter)

| Theoretical | Measured | Mean ± SD | Recovery (%) |
|---|---|---|---|
| 0 | 6.9 | 2.9 ± 3.6 | n.a |
| | 1.7 | | |
| | <0.5 | | |
| 10.7 | 15.4 | 16.6 ± 1.1 | 155 |
| | 17.2 | | |
| | 17.3 | | |
| 107 | 95.2 | 94 ± 2 | 88 |
| | 94.4 | | |
| | 92.3 | | |
| 1071 | 1030 | 1070 ± 78 | 100 |
| | 1160 | | |
| | 1020 | | |
| 2141 | 1940 | 1890 ± 132 | 88 |
| | 1740 | | |
| | 1990 | | |
| 4283 | 3490 | 4537 ± 1236 | 106 |
| | 5900 | | |
| | 4220 | | |

Filters to which a certain amount of lipid-based cisplatin was applied (performed in triplicate) were dried overnight prior to placement in tubes. An amount of 1.5 ml per sample was sent for determination of platinum levels by ICP-MS. The first 2 columns show the actual concentrations added to the filters and the concentrations of lipid-based cisplatin on the filters measured by ICP-MS consecutively. The third column shows the mean±standard deviation of the measurements (the SD for measurements of the blanks (σ) was used to calculate the LOD and LOQ). Recovery is stated as the percentage of the actual value divided by the measured value and listed in the last column.

Solution based cisplatin standards were prepared at concentrations of 9.75×10⁻¹, 9.75×10⁻², and 9.75×10⁻³ mg/ml cisplatin. These data concerning the solutions of standard and liposomal cisplatin are presented in Table 2.

TABLE 2
Evaluation of ICP-MS in measuring platinum content of control samples
using standard or lipid-based platinum compound solutions.

| Mean Pt levels ± SD (ng/liter) | | |
|---|---|---|
| Theoretical | Measured | Recovery (%) |
| 10.7* | 15.0 ± 0.85 | 140 |
| 107* | 107.9 ± 12.6 | 101 |
| 1071* | 1004 ± 9.19 | 94 |
| 2141* | 2063 ± 148 | 96 |
| 4283* | 3923 ± 200 | 92 |
| 11** | 14.7 ± 0.76 | 134 |
| 105** | 108 ± 25 | 103 |
| 1055** | 896 ± 63 | 85 |
| 2109** | 1980 ± 36 | 94 |
| 4218** | 3567 ± 226 | 85 |

*Lipid-based cisplatin formulation.
**Standard cisplatin.

Residues of lipid-based cisplatin on filters were extracted with a solvent comprised of a 0.9% NaCl (weight/volume) in an n-propanol/water 75:25 (volume/volume) mixture.

Control samples were prepared in triplicate. Three mL of extraction solvent were added to each of the 48 tubes of controls and the 9 tubes of the samples. The samples were allowed to stand overnight at room temperature after which 1.5 ml was removed from each tube and placed in polypropylene vials, sealed, and shipped to an outside contract laboratory (Elemental Research Inc., Vancouver, Canada) for determination of platinum levels. Determination of platinum levels in the solution extracts of the filters was performed with an ICP-MS. Previous studies confirm that ICP-MS analysis is a sensitive method to quantitate the amount of platinum in a given sample. Falter, R. et al., Sci. Total Environ., 225: 167-176, 1999; Ghezzi, A. et al., J. Inorg. Biochem., 98: 73-78, 2004; Hann, S. et al., Anal. Bioanal. Chem., 381: 405-412, 2005; Parent, M. et al., Anal. Bioanal. Chem., 354: 664-667, 1996.

Solution extracts of the 9 filter samples and 48 controls were assayed for platinum content by ICP-MS. The 18 samples listed in Table 1 represent the lipid-based cisplatin controls on filter. With the exception of the 10.7 ng/ml sample, the recoveries varied between 88 and 106%. For the purposes of this work, the limit of detection (LOD) is defined based on data from the blanks, as described in the guidelines from the international conference on harmonization of technical requirements for registration of pharmaceuticals for human use:
LOD=3.3σ·S⁻¹ (1)
Where σ is the standard deviation for measurement of the blanks, and S is the slope of the standard curve (measured divided by theoretical concentration). International Conference on Harmonization (ICH) of Technical Requirements for Registration of Pharmaceuticals for Human Use. Harmonised Tripartite Guideline: Validation of Analytical Procedures: Methodology Q2B. 1996. Ref Type: Conference Proceeding.

Similarly, the limit of quantitation (LOQ) is defined as:
LOQ=10σ·S⁻¹ (2)

The standard deviation for measurement of the blanks (performed in triplicate) was 3.6 (Table 1) and the slope of the standard curve (S) was 1.038 (FIG. 2). After calculation the LOD is 11.4 ng/ml or 34.3 ng in 3 ml of extraction solvent, and the LOQ is 34.6 ng/ml or 103.9 ng in 3 ml of extraction solvent.

II.C. Results and Discussion

The first set of filters sampled air during patient dosing (condition one) and the second series sampled air during the time immediately following cessation of dosing (condition two). The cumulative times for both sets of filters were 832 min and 245 min, respectively. The nebulizer filled with 7 mL of lipd-based cisplatin was run to dryness, without a patient present, in 23 minutes (condition 3). Table 3 shows the air concentrations collected at the clinical study site. Pt levels were determined by using ICP-MS to analyze the solution extracts of the 9 filter samples. All of the samples collected in the study were significantly below the LOQ (=103.9 ng/ml).

The legal airborne permissible exposure limit (PEL) for cisplatin is 2·10⁻³ mg/m³ and was established by the Occupational Safety and Health Administration (OSHA). This value is averaged over an eight-hour work shift and is equivalent to 2 ng·L⁻¹ (a maximal concentration of 2 ng cisplatin per liter of air in the room during 480 consecutive minutes is permitted). In fact the PEL is a time weighted average (TWA=air concentration×time of exposure in hours divided by 8 hours). Because all the measured Platinum levels were below the LOQ (BLQ), we used the LOQ (=103.9 ng·mL⁻¹) itself to calculate the air concentrations. In stage 1, during patient dosing, all calculated air concentrations were indistinguishable from the blanks, indicating that little or no cisplatin was able to escape the demistifier during approximately 14 hours of patient dosing. For instance, if the highest air concentration is taken, calculated for this stage (air concentration is LOQ divided by the product of mean airflow and sampling time, see Table 3), i.e. <1.00 ng·L⁻¹, the TWA will be <1.73 ng·L⁻¹. If the LOD (=34.3 ng·mL⁻¹) is used instead of the LOQ in the calculation, the safety margin is at least 3.5-fold. Considering the fact that the healthcare worker spends only a small portion of their time in the room, it is clear that the demistifier provides excellent protection during patient dosing.

TABLE 3
Air concentrations at the clinical site, measured
by ICP-MS of solution extracts of air filters.

| | Pt level | Mean air flow | Sampling time | Air conc. | TWA |
|---|---|---|---|---|---|
| Filter | (ng) | (l/min) | (min) | (ng/l) | (ng/l) |
| 1A | BLQ* | 0.145 | 832 | <0.86 | <1.49 |
| 1B | BLQ | 0.205 | 832 | <0.61 | <1.06 |
| 1C | BLQ | 0.125 | 832 | <1.00 | <1.73 |
| 2A | BLQ | 0.155 | 245 | <2.74 | <1.40 |
| 2B | BLQ | 0.190 | 245 | <2.23 | <1.14 |
| 2C | BLQ | 0.115 | 245 | <3.69 | <1.88 |
| 3A | BLQ | 0.145 | 23 | <31.15 | <1.49 |
| 3B | BLQ | 0.205 | 23 | <22.04 | <1.05 |
| 3C | BLQ | 0.125 | 23 | <36.14 | <1.73 |

BLQ = below limit of quantitation (LOQ = 103.9 ng · 3 mL⁻¹). Slope of the standard curve S = 1.038.

This table shows the 9 filters that were used at the clinical site. Series 1 contain the filters used during patient dosing. Series 2 contain the filters used directly after the patient has left the tent. Series 3 contain the filters used when 7 mL of drug product was nebulized to dryness in the tent without a patient being present. The airflow through each filter was measured. Sampling times were recorded. Air concentrations of cisplatin were calculated from mean airflow and Pt levels. TWA=time weighted average.

The air concentrations measured while taking the patient from the tent (i.e. when the integrity of the tent has been compromised) are also below the LOQ. Given the shorter sampling times, the upper theoretical limit in air concentrations is higher. The calculated TWA for the highest measured air concentration, i.e. <3.69 from filter 2C, is <1.88 ng·L⁻¹, which is slightly lower than the PEL. If we use the LOD instead of the LOQ, the safety margin is 3.2-fold. It should be noted that taking the patient from the tent occurs over just a few minutes time, and thus the low values reported should not be averaged over an eight hour work shift for comparison to the PEL. Thus, the current procedure for extracting patient from the tent is adequate to control potential healthcare worker exposure to aerosolized lipid-based cisplatin.

The air concentrations measured following aerosolization of 7 mL of lipid-based cisplatin in the tent without a patient were also below the LOQ. This portion of the experiment was designed to assess whether the HEPA filter would be able to effectively clear the large volume of the nebulized drug. Indeed the levels on the filter placed in the HEPA exhaust were indistinguishable from the blanks, indicating that the HEPA was able to effectively remove aerosolized droplets. This means that the demistifier can be exhausted directly into the room, and no additional ventilation precautions need be taken.

The two filters placed outside of the tent showed levels of cisplatin below the LOQ, indicating that no aerosol was able to escape under the tent skirt under these extreme ‘no-patient’ conditions. This experiment represents a non-use scenario as there was no patient to inhale most of the drug, and there was no filter on the exhalation-side of the nebulizer circuit to reduce fugitive aerosol. The PARI exhalation filter attached to the nebulizer has been shown to be >93% effective in capturing exhaled aerosol droplets. Hence, the concentration of aerosol in the air inside the tent during patient dosing is anticipated to be 2 to 3 orders of magnitude less than was found in this experiment. As noted, no evidence of lipid-based cisplatin was found outside of the tent during patient dosing, or following loss of containment when the patient leaves the tent. These experiments (conditions one and two) represent actual patient-use scenarios.

The combination of a mobile HEPA filter air cleaning system and a demistifier tent is effective at containing nebulized lipid-based cisplatin during patient treatment, both during patient dosing, and upon extraction of the patient from the tent. Importantly, the tent is completely effective at filtering lipid-based cisplatin aerosol such that no platinum exits in the air exhausted from the tent. These results indicate that no special precautions are necessary in exhausting the tent.

III. Effective and Comfortable Administration of Lipid-Based Platinum Compound Formulations

In addition to safe methods of administering lipid-based platinum compound formulations, the present invention also relates to methods of administration that increase a patient's comfort and allows uninterrupted administration. The present invention also relates to an inhalation device comprising a holder for diverting a patient's saliva away from both the exhalation filter and from entering the nebulizer.

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be read restrictively unless there is clear and convincing evidence manifesting a certain exclusion, restriction, or disclaimer.

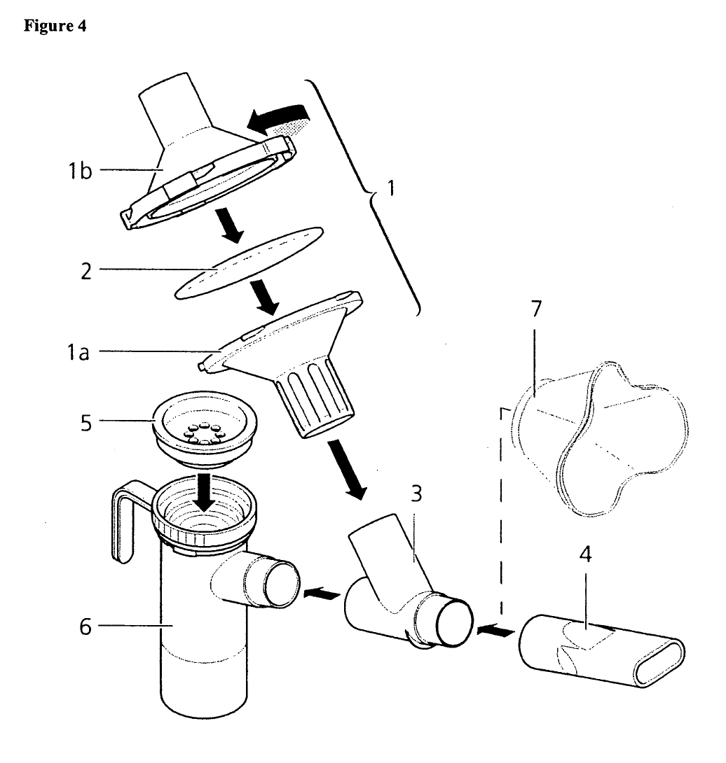

FIG. 4 depicts a blown up view of a Pari Filter/Valve Set (the “Pari device”) which is a commonly used inhalation device for the administration of chemotherapeutics. Key to the Pari device is exhalation filter 1 comprising upper part 1b, filter pad 2, and lower part 1a. Exhalation filter 1 prevents chemotherapeutics from entering the environment upon exhalation of the patient. Exhalation filter 1 attaches to y-connection 3 situated between mouth piece 4 and nebulizer 6. Inspiratory valve 5 attaches to the top of nebulizer 6. Optionally, baby mask 7 may be used with infants, and is placed between mouth piece 4 and y-connector 3 covering the infant's mouth and nose.

A serious drawback to the current inhalation devices is the position of exhalation device 1. It points upwards to prevent the patient's saliva from entering and contaminating the filter. This design however does not account for where the saliva can go. Often the answer is into the nebulizer device. An inhalation device that prevents contamination of both the exhalation filter and the drug remaining in the nebulizer from saliva is needed.

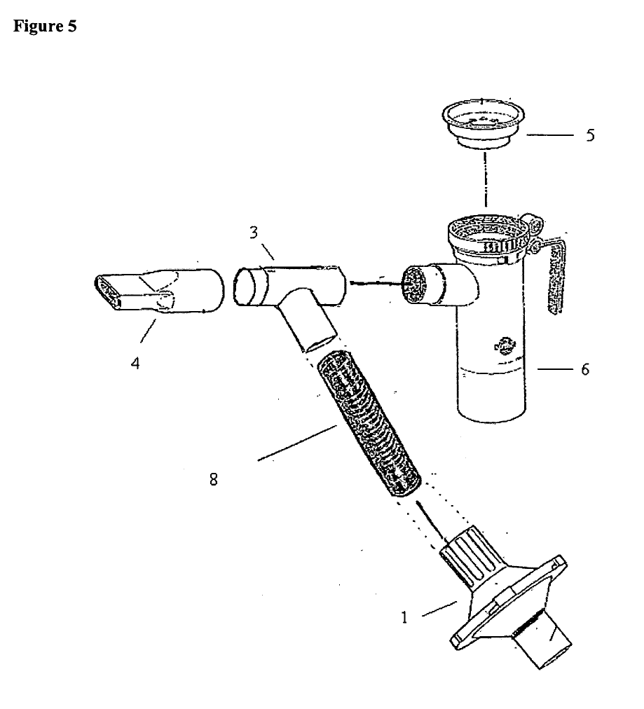

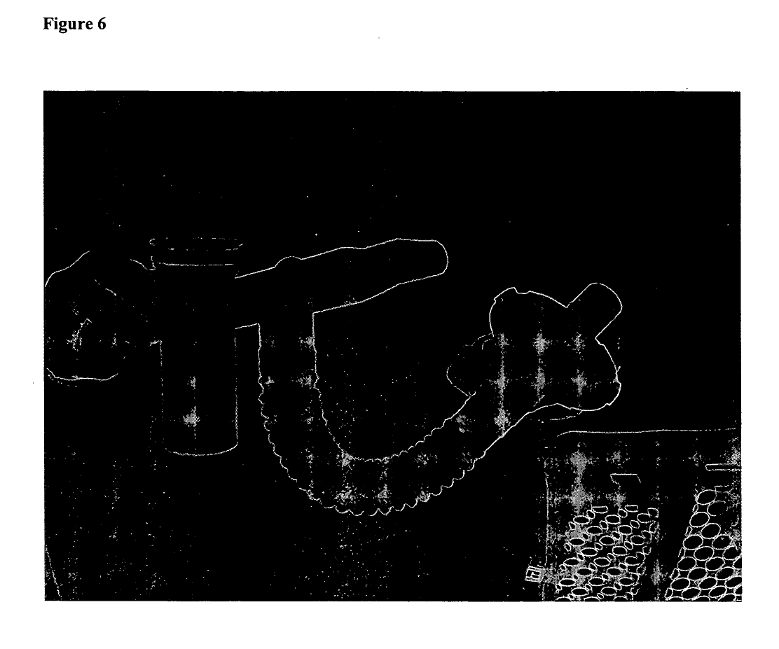

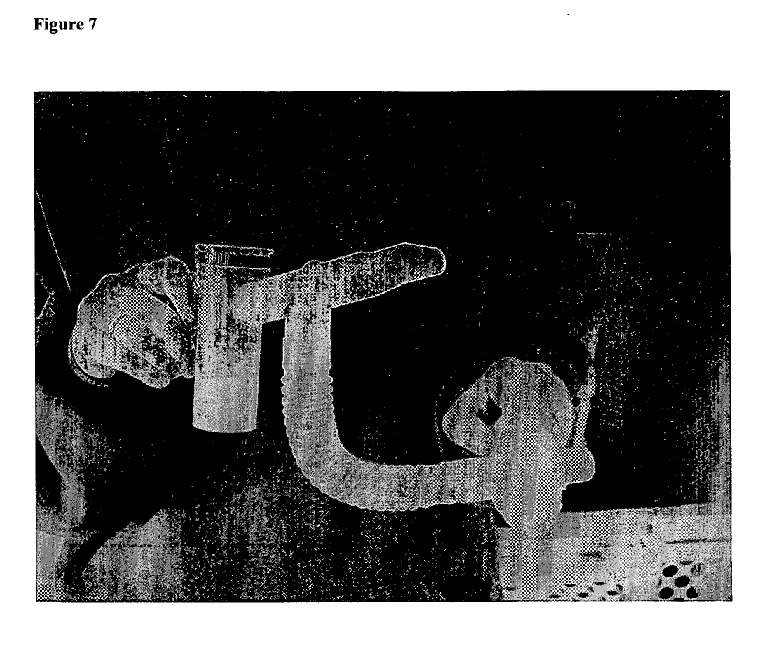

FIG. 5 discloses one embodiment of the present invention where the problem of saliva entering the nebulizer or returning to the patient is solved by inserting holder 8, which in this embodiment is a section of tubing, between y-connector 3 and exhalation filter 1. Important to this embodiment is the disposition of holder 8 and exhalation filter 1. The y-connector 3 and holder 8 point downward. The other end of holder 8 and the attached exhalation filter are oriented sideways relative to the nebulizer 6 and mouth piece 4. Holder 8 first slopes downwards as depicted. The other end of holder 8 and exhalation filter 1 then curves upward or levels out to be substantially parallel to the ground. This position allows the patient's saliva to collect in holder 8 without contaminating exhalation filter 1 or entering nebulizer 6. FIG. 6 depicts a photographic image of a non-limiting example of an embodiment of the inhalation device where holder 8 first slopes downward and then curves upward. FIG. 7 depicts a photographic image of a non-limiting example of an embodiment of the inhalation device where holder 8 first slopes downward and then levels off to be substantially parallel to the ground.

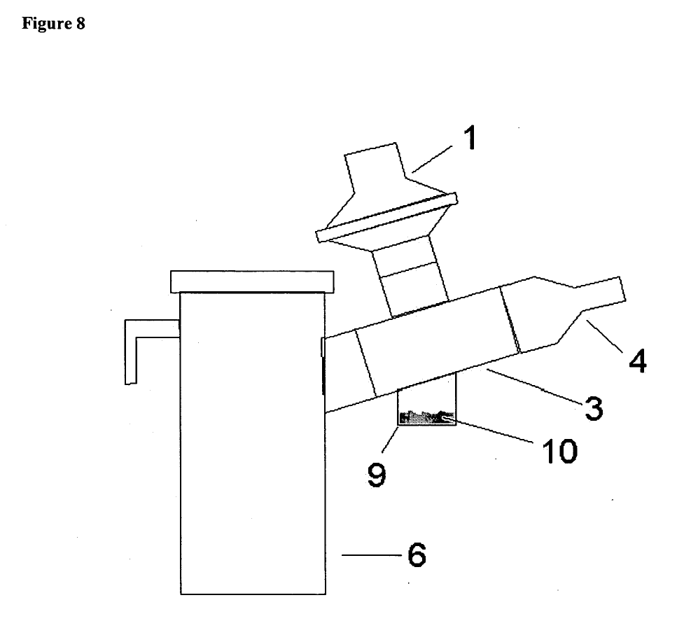

FIG. 8 depicts another embodiment of the present invention. In this embodiment y-connector 3 has been altered to comprise holder 9 in the form of a cup. Like in the previous embodiment, the holder is placed between the path of mouth piece 4 and exhalation filter 1. In this embodiment, exhalation filter 1 can remain in its normal position pointing upwards and in the same plane as nebulizer 6 and mouth piece 4. Saliva 10 will not enter exhalation filter 1 but will rather collect in holder 9 and not enter nebulizer 6 or return to the patient.

Although specific features of the invention are shown in some drawings and not in others, this is or convenience only as each feature may be combined with any or all of the other features in accordance with invention. The words “including,” “comprising,” “having,” and “with as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

IV. Lipids

The lipids used in forming the lipid-based formulations of a platinum compound or other chemotherapeutic agent may be synthetic, semi-synthetic or naturally-occurring lipids, including phospholipids, tocopherols, sterols, fatty acids, glycoproteins such as albumin, negatively-charged lipids and cationic lipids. In terms of phosholipids, they could include such lipids as egg phosphatidyl choline (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC. Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (Pls) and the phosphatidyl serines (PSs). Examples include DMPG, DPPG, DSPG, DMPA, DPPA, DSPA, DMPI, DPPI, DSPI, DMPS, DPPS and DSPS.

V. Liposomes

Liposomes are completely closed lipid bilayer membranes containing an entrapped aqueous volume. Liposomes used for the parenteral delivery of an antineoplastic compound may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilanellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

Another method of preparing liposomes is the “solvent infusion” process. Solvent infusion is a process that includes dissolving one or more lipids in a small, preferably minimal, amount of a process compatible solvent to form a lipid suspension or solution (preferably a solution) and then adding the solution to an aqueous medium containing, for example, platinum compounds. Typically a process compatible solvent is one that can be washed away in an aqueous process such as dialysis. The composition that is cool/warm cycled is preferably formed by solvent infusion, with ethanol infusion being preferred.

The process for producing lipid-based platinum compound formulations may comprise mixing a platinum compound with an appropriate hydrophobic matrix and subjecting the mixture to one or more cycles of two separate temperatures. The process is believed to form active platinum compound associations.

In aqueous solution, when the platinum compound is cisplatin, it may form large insoluble aggregates with a diameter of greater than a few microns. In the presence of a amphipathic matrix system, such as a lipid bilayer, cisplatin-lipid associations form. For example, the associations may be formed in the internal aqueous space, the hydrocarbon core region of a lipid bilayer, or the liposome interface or headgroup. During the warming cycle of the process, it is believed that cisplatin is returned to solution at a greater rate in aqueous regions of the process mixture than from the lipid-matrix. As a result of applying more than one cool/warm cycle, cisplatin accumulates further from the lipid-matrix. Without limiting the invention to the proposed theory, experimentation indicates that the cisplatin-lipid associations cause the immediate surroundings of the interfacial bilayer region to be more hydrophobic and compact. This results in a high level of entrapment of active platinum compound as cooling and warming cycles are repeated.

The process comprises combining the platinum compound with a hydrophobic matrix carrying system and cycling the solution between a warmer and a cooler temperature. Preferably the cycling is performed more than one time. More preferably the step is performed two or more times, or three or more times. The cooler temperature portion of cycle can, for example, use a temperature from about −25° C. to about 25° C. More preferably the step uses a temperature from about −5° C. to about 25° C. or from about 1° C. to about 20° C. For manufacturing convenience, and to be sure the desired temperature is established, the cooler and warmer steps can be maintained for a period of time, such as approximately from 5 to 300 minutes or 30 to 60 minutes. The step of warming comprises warming the reaction vessel to from about 4° C. to about 70° C. More preferably the step of warming comprises heating the reaction vessel to about 45° C. or to about 55° C. The above temperature ranges are particularly preferred for use with lipid compositions comprising predominantly diphosphatidycholine (DPPC) and cholesterol.

Another way to consider the temperature cycling is in terms of the temperature differential between the warmer and the cooler steps of the cycle. This temperature differential can be, for example, about 25° C. or more, such as a differential from about 25° C. to about 70° C., preferably a differential from about 40° C. to about 55° C. The temperatures of the cooler and higher temperature steps are selected on the basis of increasing entrapment of active platinum compound. Without being limited to theory, it is believed that it is useful to select an upper temperature effective substantially increase the solubility of active platinum compound in the processed mixture. Preferably, the warm step temperature is about 50° C. or higher. The temperatures can also be selected to be below and above the transition temperature for a lipid in the lipid composition.

The temperatures appropriate for the method may, in some cases, vary with the lipid composition used in the method, as can be determined by ordinary experimentation.

The platinum compound to lipid ratio seen in the lipid-based platinum formulations used in the present invention may be between about 1:5 by weight and about 1:50 by weight. More preferably the platinum compound to lipid ratio achieved is between about 1:5 by weight and about 1:30 by weight. Most preferably the platinum compound to lipid ratio achieved is between about 1:5 by weight and about 1:25 by weight.

The liposomes have a mean diameter of approximately 0.01 microns to approximately 3.0 microns, preferably in the range about 0.1 to 1.0 microns. More preferably, the mean diameter is from about 0.1 to 0.5 microns. The sustained release property of the liposomal product can be regulated by the nature of the lipid membrane and by inclusion of other excipients (e.g., sterols) in the composition.

In a preferred embodiment of the invention the liposome contains about 50 to about 100 mol % DPPC and about 0 to about 50 mol % cholesterol. More preferably, the liposome contains about 50 to about 65 mol % DPPC and about 35 to about 50 mol % cholesterol.

Liposomes can also be prepared by the methods disclosed in copending U.S. patent application Ser. No. 10/383,004, filed Mar. 5, 2003; Ser. No. 10/634,144, filed Aug. 4, 2003; Ser. No. 10/224,293, filed Aug. 20, 2002; and Ser. No. 10/696,389, filed Oct. 29, 2003, the specifications of which are incorporated herein in their entirety.

VI. Platinum Compounds

The platinum compounds that may be used in the present invention include any compound that exhibits the property of preventing the development, maturation, or spread of neoplastic cells. The platinum compounds of the kit, method, and inhalation device embodiments of the present invention need not be in lipid-based formulations. They may be free platinum compounds or in other formulations generally known in the art. For example, the platinum compound may be a free platinum compound such as Platinol. Non-limiting examples of platinum compounds include cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro(cyclohexylamine)dihydroxoplatinum(IV)), and (trans, trans, trans)bis-mu-(hexane-1,6-diamine)-mu-[diamine-platinum(II)]bis[diamine(chloro)platinum(II)]tetrachloride. In another embodiment the platinum compound is cisplatin. Depending on the environment, cisplatin may exist in a cationic aquated form wherein the two negatively charged chloride atoms have been displaced by two neutral water molecules.

In other embodiments, other therapeutic agents may be used with the platinum compounds. The other therapeutic agents may have antineoplastic properties. Non-limiting examples of antineoplastic compounds include altretamine, amethopterin, amrubicin, annamycin, arsenic trioxide, asparaginase, BCG, benzylguanine, bisantrene, bleomycin sulfate, busulfan carmustine, cachectin, chlorabucil, 2-chlorodeoxyadenosine, cyclophosphamide, cytosine arabinoside, dacarbazine imidazole carboxamide, dactinomycin, daunomycin, 3′-deamino-3′-morpholino-13-deoxo-10-hydroxycarminomycin, 4-demethoxy-3-deamino-3-aziridinyl-4-methylsulphonyl-daunorubicin, dexifosfamide, dexamethasone, diarizidinylspermine, dibromodulcitol, dibrospidium chloride, 1-(11-dodecylamino-10-hydroxyundecyl)-3,7-dimethylxanthine, doxorubicin, elinafide, epipodophyllotoxin, estramustine, floxuridine, fluorouracil, fluoxymestero, flutamide, fludarabine, fotemustine, galarubicin, glufosfamide, goserelin, GPX100, hydroxyurea, idarubicin HCL, ifosfamide, improsulfan tosilate, isophosphamide, interferon alfa, interferon alfa 2a, interferon alfa 2b, interferon alfa n3, interferon gamma, interleukin 2, irinotecan, irofulven, leucovorin calcium, leuprolide, levamisole, lomustine, megestrol, L-phenylalanie mustard, L-sarcolysin, melphalan hydrochloride, mechlorethamine, MEN10755, mercaptopurine, MESNA, methylprednisolone, methotrexate, mitomycin, mitomycin-C, mitoxantrone, nimustine, paclitaxel, pinafide, pirarubicin, plicamycin, prednimustine, prednisone, procarbazine, profiromycin, pumitepa, ranimuistine, sertenef, streptozocin, streptozotocin, tamoxifen, tasonermin, temozolomide, 6-thioguanine, thiotepa, tirapazimine, triethylene thiophosporamide, trofosfamide, tumor necrosis factor, valrubicin, vinblastine, vincristine, vinorelbine tartrate, and zorubicin.

Also included as suitable platinum compounds used in the kits, methods, and inhalation device embodiments of the present invention are pharmaceutically acceptable addition salts and complexes of platinum compounds. In cases wherein the compounds may have one or more chiral centers, unless specified, the present invention comprises each unique racemic compound, as well as each unique nonracemic compound.

In cases in which the platinum compounds have unsaturated carbon-carbon double bonds, both the cis (Z) and trans (E) isomers are within the scope of this invention. In cases wherein the neoplastic compounds may exist in tautomeric forms, such as keto-enol tautomers, such as 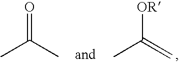
each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included as suitable platinum compounds used in the kits, methods, and inhalation device embodiments of the present invention are prodrugs of the platinum compounds. Prodrugs are considered to be any covalently bonded carriers which release the active parent compound in vivo.

VII. Therapeutic Agents

Although platinum compounds and lipid-based compositions thereof are the preferred therapeutic agents, it is important to realize that the kit, method, and inhalation device embodiments of the present invention are not limited to this class of therapeutic agents. It is envisioned by the inentors that other therapeutic agents may be used in the kit, method, and inhalation device embodiments of the present invention. Non-limiting examples of other therapeutic agents include sulfonamide, such as sulfonamide, sulfamethoxazole and sulfacetamide; trimethoprim, particularly in combination with sulfamethoxazole; a quinoline such as norfloxacin and ciprofloxacin; a beta-lactam compound including a penicillin such as penicillin G, penicillin V, ampicillin, amoxicillin, and piperacillin, a cephalosporin such as cephalosporin C, cephalothin, cefoxitin and ceftazidime, other beta-lactarn antibiotics such as imipenem, and aztreonam; a beta lactamase inhibitor such as clavulanic acid; an aminoglycoside such as gentamycin, amikacin, tobramycin, neomycin, kanamycin and netilmicin; a tetracycine such as chlortetracycline and doxycycline; chloramphenicol; a macrolide such as erythromycin; or miscellaneous antibiotics such as clindamycin, a polymyxin, and bacitracin for anti-bacterial, and in some cases antifungal, infections; a polyene antibiotic such as amphotericin B, nystatin, and hamycin; flucytosine; an imidazole or a triazole such as ketoconazole, miconazole, itraconazole and fluconazole; griseofulvin for anti-Fungal diseases such as aspergillosis, candidaisis or histoplasmosis; zidovudine, acyclovir, ganciclovir, vidarabine, idoxuridine, trifluridine, an interferon (e.g, interferon alpha-2a or interferon alpha-2b) and ribavirin for anti-viral disease; aspirin, phenylbutazone, phenacetin, acetaminophen, ibuprofen, indomethacin, sulindac, piroxicam, diclofenac; gold and steroidal anti-inflammatories for inflammatory diseases; an ACE inhibitor such as captopril, lovostatin, lipitor, clofibrate, cholestryamine, probucol, and nicotinic acid, enalapril, quinidine, procainamide, lidocaine, encainide, propranolol, esmolol, bretylium, verapimil, diltiazem, and lisinopril; the organo nitrates such as amyl nitrite, nitroglycerin and isosorbide dinitrate; the calcium channel blockers such as diltiazem, nifedipine and verapamil; the beta adrenegic antagonists such as propranolol for cardiovascular disease; a diuretic such as a thiazide; e.g., benzothiadiazine or a loop diuretic such as furosemide; a sympatholytic agent such as methyldopa, clonidine, gunabenz, guanaethidine and reserpine; a vasodilator such as hydalazine and minoxidil; a calcium channel blocker such as verapimil; an anthracycline such as doxorubicin, daunorubicin and idarubicin; a covalent DNA binding compound, a covalent DNA binding compound; a folate antagonist such as methotrexate and trimetrexate; an antimetabolite and a pyrimidine antagonist such as fluorouracil, 5-fluorouracil and fluorodeoxyuridine; an antimetabolite and a purine antagonist such as mercaptopurine, 6-mercaptopurine and thioguanine; an antimetabolite and a sugar modified analog such as cytarabine and fludarabine; an antimetabolite and a ribonucleotide reductase inhibitor such as hydoxyurea; a covalent DNA binding compound and a nitrogen mustard compound such as cyclophosphamide and ifosfamide; a covalent DNA binding compound and an alkane sulfonate such as busulfane; a nitrosourea such as carmustine; a covalent DNA binding compound and a methylating agent such as procarbazine; a covalent DNA binding compound and an aziridine such as mitomycin; a non covalent DNA binding compound; a non covalent DNA binding compound such as mitoxantrone and, bleomycin; an inhibitor of chromatin function and a topoisomerase inhibitor such as etoposide, teniposide, camptothecin and topotecan; an inhibitor of chromatin function and a microtubule inhibitor such as the vinca alkaloids including vincristine, vinblastin, vindisine, and paclitaxel, taxotere or another taxane; a compound affecting endocrine function such as prednisone, prednisolone, tamoxifen, leuprolide, ethinyl estradiol, an antibody such as herceptin; a gene such as the p-53 gene, the p 16 gene, the MIT gene, and the gene E-cadherin; a cytokine such as the interleukins, particularly, IL-1, IL-2, IL-4, IL-6, IL-8 and IL-12, the tumor necrosis factors such as tumor necrosis factor-alpha and tumor necrosis factor-beta, the colony stimulating factors such as granulocyte colony stimulating factor (G-CSF), macrophage colony stimulating factor (M-CSF) and, granulocyte macrophage colony stimulating factor (GM-CSF) an interferon such as interferon-alpha, interferon-beta 1, interferon-beta 2, and interferon-gamma; all-trans retinoic acid or another retinoid for the treatment of cancer; an immunosuppressive agent such as: cyclosporine, an immune globulin, and sulfasazine, methoxsalen and thalidoimide; insulin and glucogon for diabetes; calcitonin and sodium alendronate; morphine and related opioids; meperidine or a congener; methadone or a congener; an opioid antagonist such as nalorphine; a centrally active antitussive agent such as dexthromethrophan; tetrahydrocannabinol or marinol, lidocaine and bupivicaine for pain management; chloropromazine, prochlorperazine; a cannabinoid such as tetrahydrocannabinol, a butyrophenone such as droperidol; a benzamide such as metoclopramide for the treatment of nausea and vomiting; heparin, coumarin, streptokinase, tissue plasminogen activator factor(t-PA) as anticoagulant, antithrombolytic or antiplatelet drugs; heparin, sulfasalazine, nicotine and adrenocortical steroids and tumor necrosis factor-alpha for the treatment of inflammatory bowel disease; nicotine for the treatment of smoking addiction; growth hormone, luetinizing hormone, corticotropin, and somatotropin for hormonal therapy; and adrenaline for general anaphylaxis.

In regard to lung disease, therapeutic agents include: a methylxanthine such as theophylline; cromolyn; a beta-adrenginic agonist such as albuterol and tetrabutaline; a anticholinergic alkaloid such as atropine and ipatropium bromide; adrenocortical steroids such as predisone, beclomethasone and dexamethasone for asthma or inflammatory disease; the anti-bacterial and antifungal agents listed above, in particular this includes the use of aminoglycosides (e.g., amikacin, tobramycin and gentamycin), polymyxins (e.g., polymyxin E, colistin), carboxycillin (ticarcillin) and monobactams for the treatment of gram-negative anti-bacterial infections, for example, in cystic fibrosis patients, for the treatment of gram negative infections of patients with tuberculosis, for the treatment of gram negative infections in patients with chronic bronchitis and bronchiectasis, and for the treatment of gram negative infections in generally immuno-compromised patients; the use of pentamidine for the treatment of patients (e.g., HIV/AIDS patients) with Pneumocytis carinii infections; the use of a polyene antibiotic such as amphotericin B, nyststin, and hamycin; flucytosine; an imidazole or a triazole such as ketoconazole, miconazole, itraconazole and fluconazole; griseofulvin for the treatment of such fungal infections as aspergillosis, candidiasis and histoplasmosis, particularly those originating or disseminating to the lungs; the use of the corticosteroids and other steroids as listed above, as well as nonsteroidal anti-inflammatory drugs for the treatment of anti-inflammatory conditions in patients with lung disease (these are the specific diseases listed above in what lung disease includes); DNase, amiloride, CFTRcDNA in the treatment of cystic fibrosis; alpha-1-antitrypsin and alpha-1-antitrypsin cDNA for the treatment of emphysema; an aminoglycoside such as amikacin, tobramycin or gentamycin, isoniazid, ethambutol, rifampin and its analogs for the treatment of tuberculosis or mycobacterium infections; ribavirin for the treatment of respiratory synctial virus; the use of the anticancer agents listed above for lung cancer in particular cisplatin, carboplatin, and taxanes such as paclitaxel, and the taxanes, camptothecin, topotecin, and other camptothecins, herceptin, the p-53 gene and IL-2.

VIII. Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 50 mg per kg.

Dosage amounts are also commonly administered as mg/m² which stands for milligrams of drug (e.g. platinum compound) per body surface area. Generally, dosage amounts for platinum compounds may be about 30 mg/m² or greater, 50 mg/m² or greater, 80 mg/m² or greater, or 100 mg/m² or greater. Dosage amounts of about 80 mg/m² or greater are generally considered at the high end of tolerance, but an advantage of the present invention is that the platinum compound is administered as part of a lipid-based formulation which decreases the sub-acute toxicities of the platinum compound. It is therefore envisioned by the inventors that higher than normal dosage amounts of platinum compound may be administered to the patient without unwanted toxic side effects.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the antineoplastic compound) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

IX. Pharmaceutical Formulation

The pharmaceutical formulation of the antineoplastic compound may be comprised of an aqueous dispersion of liposomes. The formulation may contain lipid excipients to form the liposomes, and salts/buffers to provide the appropriate osmolarity and pH. The pharmaceutical excipient may be a liquid, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

X. Kits

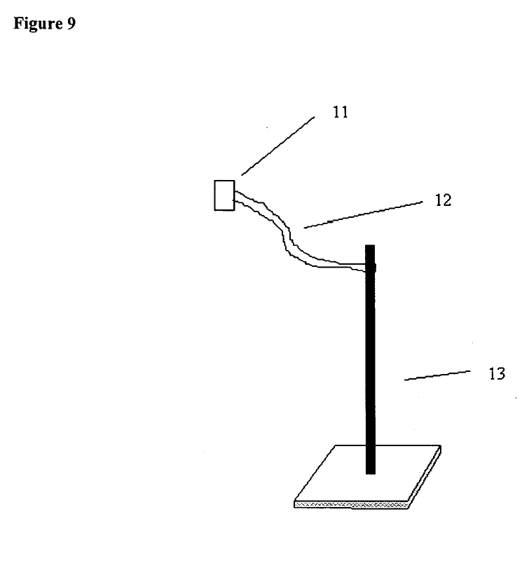

This invention also provides kits for conveniently and effectively implementing the methods of this invention. Such kits comprise any of the compounds of the present invention or a combination thereof, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use. In one embodiment, the present invention relates to a kit comprising an inhalation device comprising a lipid-based platinum compound formulation, a demistifier tent of sufficient size to at least cover a patient's head and the inhalation device, and instructions for use thereof. In a further embodiment the kit may also comprise a stand as depicted in FIG. 9 to hold the inhalation device for additional patient comfort. The stand comprises clamp 11 for securing the inhalation device. Arm 12 is preferably flexible and extends from post 13 to clamp 11. Preferably, arm 12 can also be adjusted vertically along post 13.

The following examples further illustrate the present invention, but of course, should not be construed as in any way limiting its scope.

### EXEMPLIFICATION

Materials and Methods. A lipid-based cisplatin formulation at a 1 mg/ml concentration was manufactured at Transave Inc. (Monmouth Junction, N.J., USA). A PARI LC STAR nebulizer (FIG. 4) was used for aerosol delivery. The nebulizer was connected to a PARI filter set (PARI exhalation filter-2) containing a filter pad (with permanently charged rectangular split fibers) collecting the exhaled aerosols. A study conducted to determine the integrity of this filter in monitoring the extraneous escape of product during the use of the nebulizer indicated that the collection efficiency was over 93% for twenty minutes of nebulization (after every 20 minutes of nebulization a new nebulizer with filter is used). This is an underestimation of the expected trapping efficiency during patient treatment, as extreme conditions were used in the study. The variable-pressure compressor (Easy Comp PM 50E compressor, Precision Medical Inc., Northampton, Pa., USA) was set to deliver a pressure of 30 psig for nebulization and provides an output rate of drug product of about 0.3 mL/min. For nebulized lipid-based cisplatin droplet size, the Mass Median Aerodynamic Diameter (MMAD) was found to be ˜3.7 microns and Geometric Standard Deviation (GSD) was found to be ˜1.9 MMAD and was measured by cascade impaction using a Next Generation Pharmaceutical Impactor (NGI Model 170; MSP Corp., Shoreview, Minn., USA) which operated at 5° C. and 15 L/min flow rate. The mean particle size of the lipid-based cisplatin liposomes was typically less than 0.5 microns and determined by laser diffraction/scattering. Air sampling cartridges consisted of a filter holder (47 mm with 0.25 inch connectors) with a glass fiber filter inside (47 mm, type A/E, Pall Life Sciences, Ann Arbor, Mich., USA) and were attached via a 3 meter length of Tygon vacuum tubing (0.25 inch internal diameter) to a 4-way tubing connector which again was connected to a vacuum pump with a line to the exhaust system. The collection filters were the same filters as those used at the clinical site and in the preparation of standards for quantitation.

### Example 1

Preparation of lipid-based platinum compound formulation. The lipid-based platinum compound formulations can be prepared according to the techniques disclosed in U.S. Pat. No. 6,793,912; U.S. Published Patent Application Nos. 2005/0107287 A1; 2004/0101553 A1; 2003/0059375 A1; and U.S. patent application Ser. Nos. 11/084,070; 11/135,625; the entirety of which are incorporated herein. A typical preparation where the platinum compound is cisplatin is given.

Seventy mg of DPPC and 28 mg of cholesterol were dissolved in 1 mL of ethanol and added to 10 mL of 4 mg/mL cisplatin in 0.9% saline solution. An aliquot (50%) of the sample was treated by 3 cycles of cooling to 4° C. and warming to 50° C. The aliquot, in a test tube, was cooled by refrigeration, and heated in a water bath. The resulting unentrapped cisplatin (free cisplatin) was washed by dialysis. The remainder of the sample was not treated by temperature cycles and directly washed by dialysis.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A kit for administering a lipid-based platinum compound formulation via inhalation comprising: an inhalation device comprising a lipid-based platinum compound formulation, a demistifier tent of sufficient size to at least cover a patient's head and the inhalation device, and instructions for use thereof.

2. The kit of claim 1, further comprising a stand for holding the inhalation device.

3. The kit of claim 1, wherein the platinum compound is selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

4. The kit of claim 1, wherein the platinum compound is cisplatin.

5. The kit of claim 1, wherein the lipid-based platinum compound formulation comprises a liposome.

6. The kit of claim 1, wherein the lipid-based platinum compound formulation comprises a liposome having a mean diameter of 0.01 microns to 3.0 microns.

7. The kit of claim 1, wherein the lipid is a mixture of a phospholipid and sterol.

8. The kit of claim 1, wherein the lipid is selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS); distearoylphosphatidylserine (DSPS), and mixture thereof.

9. The kit of claim 1, wherein the lipid is a mixture of DPPC and cholesterol.

10. The kit of claim 1, wherein the lipid is a mixture of DPPC from 50 to 65 mol % and cholesterol from 35 to 50 mol %.

11. The kit of claim 1, wherein the demistifier tent is large enough to cover the patient's head and inhalation device.

12. The kit of claim 1, wherein the demistifier is large enough to contain the patient.

13. The kit of claim 1, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

14. The kit of claim 1, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

15. A method of administering a lipid-based platinum compound formulation via inhalation to a patient in need thereof comprising administering the formulation to the patient while the patient is partially or wholly within a demistifier tent.

16. The method of claim 15, wherein the platinum compound is selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

17. The method of claim 15, wherein the platinum compound is cisplatin.

18. The method of claim 15, wherein the lipid-based platinum compound formulation comprises a liposome.

19. The method of claim 15, wherein the lipid-based platinum compound formulation comprises a liposome having a mean diameter of 0.01 microns to 3.0 microns.

20. The method of claim 15, wherein the lipid is a mixture of a phospholipid and sterol.

21. The method of claim 15, wherein the lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

22. The method of claim 15, wherein the lipid is a mixture of DPPC and cholesterol.

23. The method of claim 15, wherein the lipid is a mixture of DPPC from 50 to 65 mol % and cholesterol from 35 to 50 mol %.

24. The method of claim 15, wherein the demistifier tent is large enough to cover the patient's head and inhalation device.

25. The method of claim 15, wherein the demistifier is large enough to contain the patient.

26. The method of claim 15, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

27. The method of claim 15, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

28. A method of increasing the safety of administering a lipid-based platinum compound formulation via inhalation for a person comprising administering the formulation to a patient while the patient is partially or wholly within a demistifier tent.

29. The method of claim 28, wherein the platinum compound is selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro (cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

30. The method of claim 28, wherein the platinum compound is cisplatin.

31. The method of claim 28, wherein the lipid-based platinum compound formulation comprises a liposome.

32. The method of claim 28, wherein the lipid-based platinum compound formulation comprises a liposome having a mean diameter of 0.01 microns to 3.0 microns.

33. The method of claim 28, wherein the lipid is a mixture of a phospholipid and sterol.

34. The method of claim 28, wherein the lipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidyl choline (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidyl choline (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

35. The method of claim 28, wherein the lipid is a mixture of DPPC and cholesterol.

36. The method of claim 28, wherein the lipid is a mixture of DPPC from 50 to 65 mol % and cholesterol from 35 to 50 mol %.

37. The method of claim 28, wherein the demistifier tent is large enough to cover the patient's head and inhalation device.

38. The method of claim 28, wherein the demistifier is large enough to contain the patient.

39. The method of claim 28, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to cover the patient's head and inhalation device.

40. The method of claim 28, wherein the lipid-based platinum compound formulation comprises a liposome having a mean diameter of 0.01 microns to 3.0 microns, and cisplatin, wherein the lipid is a mixture of DPPC from 50 to 65 mol % and cholesterol from 35 to 50 mol %, and wherein the demistifier tent is large enough to contain the patient.

41. An inhalation device for administering a therapeutic agent to a patient comprising a mouth piece, a nebulizer, and a holder for preventing the patient's saliva from entering the nebulizer or returning to the patient during administration.

42. The inhalation device of claim 41, further comprising an exhalation filter.

43. The inhalation device of claim 42, wherein the holder is a section of tubing placed between the mouth piece and the exhalation filter such that the section of tubing slopes downwards and then curves upwards, or the section of tubing slopes downward and then levels out to be substantially parallel to the ground.

44. The inhalation device of claim 41, wherein the holder is a cup placed between the mouth piece and the nebulizer.

45. The inhalation device of claim 44, wherein the cup has a removable bottom for emptying the saliva.

46. The inhalation device of claim 41, wherein the therapeutic agent comprises a platinum compound, antiviral compound, antibacterial compound, or antifungal compound.

47. The inhalation device of claim 41, wherein the therapeutic agent comprises a chemotherapeutic agent.

48. The inhalation device of claim 41, wherein the therapeutic agent comprises a platinum compound.

49. The inhalation device of claim 41, wherein the therapeutic agent comprises a lipid-based platinum compound formulation.

50. The inhalation device of claim 41, wherein the therapeutic agent comprises a platinum compound selected from the group consisting of: cisplatin, carboplatin(diammine(1,1-cyclobutanedicarboxylato)-platinum(II)), tetraplatin(ormaplatin)(tetrachloro(1,2-cyclohexanediamine-N,N′)-platinum(IV)), thioplatin(bis(O-ethyldithiocarbonato)platinum(II)), satraplatin, nedaplatin, oxaliplatin, heptaplatin, iproplatin, transplatin, lobaplatin, cis-aminedichloro(2-methylpyridine)platinum, JM118 (cis-amminedichloro(cyclohexylamine)platinum(II)), JM149 (cis-amminedichloro(cyclohexylamine)-trans-dihydroxoplatinum(IV)), JM216 (bis-acetato-cis-amminedichloro(cyclohexylamine)platinum(IV)), JM335 (trans-amminedichloro (cyclohexylamine)dihydroxoplatinum(IV)), (trans, trans, trans)bis-mu-(hexane-1,6-diamine)-mu-[diamine-platinum(II)]bis[diamine(chloro)platinum(II)]tetrachloride, and mixture thereof.

51. The inhalation device of claim 41, wherein the therapeutic agent comprises cisplatin.

52. The inhalation device of claim 49, wherein the platinum compound is cisplatin.

53. The inhalation device of claim 49, wherein the lipid-based platinum compound formulation comprises a liposome.

54. The inhalation device of claim 49, wherein the lipid-based platinum compound formulation comprises a liposome having a mean diameter of 0.01 microns to 3.0 microns.

55. The inhalation device of claim 49, wherein the lipid is a mixture of a phospholipid and sterol.

56. The inhalation device of claim 49, wherein the lipid is selected from the group consisting of: egg phosphatidyl choline (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI, hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1, 2-bis(oleoyloxy)-3-(trimethylammonio)propane (DOTAP), phosphatidyl-glycerols (PGs), phosphatidic acids (PAs), phosphatidylinositols (Pls), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixture thereof.

57. The inhalation device of claim 49, wherein the lipid is a mixture of DPPC and cholesterol.

58. The inhalation device of claim 49, wherein the lipid is a mixture of DPPC from 50 to 65 mol % and cholesterol from 35 to 50 mol %.

59. The inhalation device of claim 49, wherein the lipid-based platinum compound formulation comprises a liposome having a mean diameter of 0.01 microns to 3.0 microns, and cisplatin, and wherein the lipid is a mixture of DPPC from 50 to 65 mol % and cholesterol from 35 to 50 mol %.
